(19) United States
(12) Patent Application Publication
(10) Pub. No.: US 2004/0214809 A1
(43) Pub. Date: Oct. 28, 2004

(54) Stabilized silver-ion sulfite complex compositions and methods

(75) Inventor: Christopher Capelli, Pittsburgh, PA (US)

Correspondence Address: Peter G. Carroll, MEDLEN & CARROLL, LLP, Suite 350, 101 Howard Street, San Francisco, CA, 94105, US

(73) Assignee: BioInterface Technologies, Inc.

(21) Appl. No.: 10/421,583

(22) Filed: Apr. 23, 2003

(51) Int. Cl. A61K 31/555; A61K 31/28; C07F 1/10

(52) U.S. Cl. 514/184; 514/495; 556/110

(57) ABSTRACT

The present invention relates to novel stabilized silver ion complexes having improved stability in aqueous solutions. The described stabilized silver-ion complex compositions comprise a silver-thiosulfate ion complex, a sulfite stabilizing agent either with or without a sulfite preservation agent. These carrier-free aqueous sulfite stabilized silver thiosulfate ion complex compositions have improved stability when exposed to an aqueous environment for extended periods. Consequently, these compositions have improved antibacterial, anti-viral and/or antifungal activity. These stabilized compositions can be used to coat a wound dressing, an ostomy appliance, an incontinence device, or other medical devices and impart long-term antimicrobial activity.

### FIELD OF INVENTION

The present invention relates to silver ion compositions and processes for making such compositions effective antibacterial, anti-viral and/or antifungal agents. In one embodiment, the invention relates to a method of producing silver thiosulfate ion compositions, coating medical devices comprising such compositions. In a preferred embodiment, the present invention relates to carrier-free aqueous silver thiosulfate complexes stabilized by sulfite ions.

### BACKGROUND

Topical antimicrobials are currently prescribed by healthcare providers to prevent and treat a variety of serious skin infections such as impetigo, infected diabetic ulcers, venous stasis ulcers, infected surgical wounds, burns, acne, psoriasis and other topical infections. Increasingly, topical antimicrobials that contain antibiotics are not effective against microbes which have developed drug resistance (i.e., antibiotic-resistant microbes).

Drug resistance is usually caused by a mutation within the microbe. When a colony of microbes is subjected to a dose of an antimicrobial, most of the bacteria die. However, occasionally some microbes, by chance, harbor mutant genes that render them resistant to the antimicrobial drug. Not only do these bacteria survive the antimicrobial treatment, but they transfer their “drug resistant” genes to their progeny (one bacterium can leave approximately 17,000,000 offspring within 24 hours). As a result, a specific antibiotic or antimicrobial used to treat an infection caused by that microbe may no longer be effective. Furthermore, once a microbe develops resistance to a specific antimicrobial, there is the possibility that the microbe will concomitantly be resistant to the entire class of antimicrobials.

Certain antimicrobials, especially antibiotics, are becoming increasingly ineffective due to the rapid increase in drug-resistant forms of microbes. For example, mupirocin ointment (Bactroban®, SmithKline Beecham) is a topical antimicrobial used most frequently for treatment of impetigo. Mupirocin has been shown to be highly effective against Staphylococcus aureus, S. epidermidis, S. saprophyticus, and Streptococcus pyogenes. Unfortunately, microbes frequently develop drug resistance to mupirocin.

Presently-available silver-based antimicrobial compositions have limited applications, often quickly lose their antimicrobial efficacy, and are frequently unstable. What is needed are pharmaceutical compositions useful in the prevention and treatment of infections and diseases which comprise an antimicrobial agent and one or more medicinal agents and which remain antimicrobially active.

### SUMMARY OF THE INVENTION

The present invention relates to compositions of silver ion complexes and processes for making such compositions effective antibacterial, antiviral and antifungal agents. In one embodiment, the invention relates to a method of producing silver thiosulfate ion complexes. In another embodiment, the invention contemplates coating medical devices comprising such compositions. In yet another embodiment, the present invention relates to carrier-free aqueous silver thiosulfate complexes stabilized by sulfite ions. Preferably, the silver thiosulfate ion complexes are stable in an aqueous environment for extended periods of time.

One aspect of the present invention contemplates a composition, comprising: a) a silver ion complex; b) a sulfite capable of stabilizing said complex; and c) an agent capable of preserving said sulfite stabilizing agent. In one embodiment, said silver ion complex comprises thiosulfate. In one embodiment, said complex is carrier-free. In another embodiment, said composition is aqueous. In one embodiment, said sulfite is selected from the group consisting of sodium sulfite, potassium sulfite, sodium bisulfite, potassium bisulfite, sodium metabisulfite, and potassium metabisulfite. In one embodiment, said agent is selected from the group consisting of glycerol, methanol, ethanol, propanol, butanol and polyvinylalcohol. In another embodiment, said agent is selected from the group consisting of methylamine, ethylamine, propylamine, butylamine and tri-hydroxymethylaminomethane.

Another aspect of the present invention contemplates a composition, comprising: a) a carrier-free stabilized silver thiosulfate ion complex; and b) a sulfite capable of stabilizing said complex, wherein the stability of said complex in an aqueous solution is improved. In one embodiment, said sulfite is selected from the group consisting of sodium sulfite, potassium sulfite, sodium bisulfite, potassium bisulfite, sodium metabisulfite, and potassium metabisulfite. In one embodiment, said composition further comprises an agent selected from the group consisting of glycerol, methanol, ethanol, propanol, butanol and polyvinylalcohol. In another embodiment, said composition further comprises an agent selected from the group consisting of methylamine, ethylamine, propylamine, butylamine and tri-hydroxymethylaminomethane.

Another aspect of the present invention contemplates a method, comprising: a) providing, i) a patient exhibiting symptoms of a microbial infection; and ii) an aqueous carrier-free sulfite stabilized silver thiosulfate ion complex; and b) administering said thiosulfate ion complex to said patient under conditions that at least one symptom of said microbial infection is reduced. In one embodiment, said microbial infection is selected from the group consisting of bacterial, viral and fungal.

Another aspect of the present invention contemplates a method, comprising: a) providing, i) a medical device; and ii) an aqueous carrier-free sulfite stabilized silver thiosulfate ion complex; and b) coating said medical device with said silver thiosulfate complex under conditions that said complex exhibits antimicrobial activity. In one embodiment, said coating is hydrophilic. In one embodiment, said antimicrobial activity is selected from the group consisting of antibacterial, antiviral and antifungal. In one embodiment, said medical device is selected from the group consisting of a wound dressing, an ostomy appliance, an incontinent device and other medical devices.

Another aspect of the present invention contemplates a method, comprising: a) providing a malodorous silver thiosulfate ion complex; and b) adding a sulfite stabilizing agent and a sulfite preservation agent to said silver thiosulfate ion complex under conditions that said malodor is reduced.

Another aspect of the present invention contemplates a method, comprising: a) providing; i) a silver thiosulfate ion complex in an aqueous solution, ii) a sulfite and iii) a solvent; and b) adding said sulfite and said solvent to said aqueous solution to create a biphasic separation. In one embodiment, said solvent is acetone.

Another aspect of the present invention contemplates an apparatus comprising: a) a medical device at least partially coated with a composition, said composition comprising i) a carrier-free silver thiosulfate ion complex and ii) a sulfite capable of stabilizing said complex. In one embodiment, said composition is hydrophilic. In one embodiment, said composition has antimicrobial activity. In another embodiment, said antimicrobial activity is selected from the group consisting of antibacterial, antiviral and antifungal. In one embodiment, said medical device is selected from the group consisting of medical implants, a wound care devices, body cavity and personal protection devices. In another embodiment, said medical device is selected from the group consisting of sutures and prosthetic implants

Another aspect of the present invention contemplates a composition comprising an anhydrous polymer matrix, wherein said matrix comprises: i) a carrier-free silver thiosulfate ion complex and ii) a sulfite capable of stabilizing said complex. In one embodiment, said sulfite is selected from the group consisting of sodium sulfite, potassium sulfite, sodium bisulfite, potassium bisulfite, sodium metabisulfite, and potassium metabisulfite. In one embodiment, said composition further comprises an agent selected from the group consisting of glycerol, methanol, ethanol, propanol, butanol and polyvinylalcohol. In another embodiment, said composition further comprises an agent selected from the group consisting of methylamine, ethylamine, propylamine, butylamine and tri-hydroxymethylaminomethane.

Another aspect of the present invention contemplates a method, comprising: a) providing; i) a catheter; and ii) a composition comprising an anhydrous polymer matrix, said matrix comprising a carrier-free silver thiosulfate ion complex and a sulfite capable of stabilizing said complex; and b) at least partially coating said catheter with said composition. In one embodiment, said catheter is a urinary catheter. In another embodiment, said catheter is a male external urine catheters.

Definitions

To facilitate understanding of the invention set forth in the disclosure that follows, a number of terms are defined below.

As used herein, the term “topically” means application to the surface of the skin, mucosa, viscera, etc.

As used herein, the term “topically active drugs” indicates a substance or composition which elicits a pharmacologic response at the site of application but which is not necessarily an antimicrobial agent.

As used herein, the term “systemically active drugs” is used broadly to indicate a substance or composition which will produce a pharmacologic response at a site remote from the point of application.

As used herein, the term “medical devices” includes any material or device that is used on, in, or through a patient's body in the course of medical treatment for a disease or injury. Medical devices include, but are not limited to, such items as medical implants, wound care devices, drug delivery devices, and body cavity and personal protection devices. The medical implants include, but are not limited to, urinary catheters, intravascular catheters, dialysis shunts, wound drain tubes, skin sutures, vascular grafts, implantable meshes, intraocular devices, heart valves, and the like. Wound care devices include, but are not limited to, general wound dressings, biologic graft materials, tape closures and dressings, and surgical incise drapes. Drug delivery devices include, but are not limited to, drug delivery skin patches, drug delivery mucosal patches and medical sponges. Body cavity and personal protection devices, include, but are not limited to, tampons, sponges, surgical and examination gloves, and toothbrushes. Birth control devices include, but are not limited to, IUD's and IUD strings, diaphragms and condoms.

The term “silver thiosulfate ion complex” as used herein, refers to silver-containing materials obtained by adding a silver halide to an aqueous solution and then adding a thiosulfate salt to the solution. Preferably, the silver complexes of the present invention are derived from the complexation of silver cations from silver halides with anions from a sodium thiosulfate sal. In one embodiment, the molar ratio of thiosulfate anions to silver cations is preferably at least 1:1 and more preferably at least 1.3:1. It is desirable that the silver thiosulfate ion complexes are solid and essentially pure, i.e., they do not contain significant amounts of waste salts or other substances that interfere with their antimicrobial activity; in addition, they do not require carrier particles.

The term “stabilized” as used herein refers to any silver thiosulfate complex that, when redissolved in an aqueous solution, is more resistant to degradation then silver thiosulfate complexes made without a stabilizing agent (i.e., for example, a sulfite ion).

The term “sulfite-stabilized” silver thiosulfate ion complex, as used herein refers to any compound containing sulfite that, when in association with a silver thiosulfate ion complex prevents the appearance of marked degradation for at least 72 hours in an aqueous solution at 50° C.

The term “preservative agent” as used herein, refers to any compound that prolongs the ability of a sulfite compound to prevent the appearance of marked degradation.

The term “marked degradation” as used herein, refers to the appearance of a significant amount of black preciptiation in a solution containing a silver ion complex.

### DETAILED DESCRIPTION OF THE INVENTION

The present invention relates to silver ion compositions and processes for making such compositions effective antibacterial, anti-viral and/or antifungal agents. In one embodiment, the invention relates to a method of producing silver thiosulfate ion compositions, coating medical devices comprising such compositions. In a preferred embodiment, the present invention relates to carrier-free aqueous silver thiosulfate complexes stabilized by sulfite ions. Preferably, the sulfite ions are preserved in the presence of sulfite preservation agents.

The present invention relates to sulfite stabilized silver thiosulfate ion complexes that have improved water stability (i.e., maintaining antimicrobial activity in an aqueous environment). More particularly, the present invention describes sulfite stabilized silver thiosulfate ion complexes comprising a silver thiosulfate ion complex and a sulfite stabilizing agent, wherein said stabilized silver-ion complex composition is carrier-free and has increased stability when dissolved in an aqueous solution. Still further, the present invention describes sulfite stabilized silver thiosulfate ion complexes comprising a silver-thiosulfate ion complex, a sulfite stabilizing agent and a preservative for said sulfite stabilizing agent. These latter stabilized silver thiosulfate ion complex compositions have additional improved water stability. These compositions are useful to produce compositions that antibacterial, anti-viral and/or antifungal activity. These stabilized compositions can be used to produce medical devices, such as, but not limited to, a wound dressing, an ostomy appliance, an incontinence device, and the like.

The antiseptic activity of silver compounds is a known property. The bacteriostatic and fungistatic effect is caused by the silver ion and a simple compound which has been used clinically is for instance silver nitrate. Silver nitrate in concentrations of 0.5-1% in water shows disinfectant properties and is used for preventing infections in burns or for prophylaxis of neonatal conjunctivitis. For another silver compound, silver sulfadiazine, the antibacterial effect of the sulfadiazine molecule is further enhanced by the complexation with the disinfecting silver ion. In contrast to the silver nitrate, the solubility of the silver sulfadiazine complex is low and hence, both of the two active parts are only present in solution in low concentrations but may be present over a longer period of time before being washed out at site to be treated. The silver sulfadiazine is intensively used in the treatment of wounds, in particular burns, under the trademarks Silvadene® and Flamazine®. Silver-protein combinations are yet other antiseptic formulations which have been used in low concentrations as eye drops.

Bacteriostatic silver ion compositions are marketed in various medical devices. One example is a wound dressing having an activated charcoal cloth dressing (Actisorb®, Johnson & Johnson). Another example is a wound dressing of modified pigskin impregnated with a soluble silver compound intended for treatment of burns (EZ-Derm®, Genetic Laboratories).

A specific advantage in using the silver ion as bacteriostatic agent is the general lack of formation of bacterial tolerance or resistance to the compound. This is in contrast to many types of antibiotics (i.e., development of “antibiotic resistance”). A major drawback of using ionic silver for bacteriostatic purposes is the appearance of a dark stain subsequent to the chemical reduction of the silver ion to free silver. Such staining has been reported to give potentially permanent pigmentation of the skin, the so-called argyria. It is commonly recognized that silver containing compounds will discolor under the influence of light and or heat. Additionally, radiation sterilization protocols may lead to an unsatisfactory change of the color of a silver composition in which it is comprised, irrespective of the use in a solution, cream or gel or a medical device. These phenomenon make silver antimicrobial agents least preferred when contemplating sterilization of medical devices. Furthermore, such medical or cosmetic products often comprise antibacterial compositions wherein discoloration is highly undesirable or unacceptable to the user.

Recently, photostable silver-based antimicrobial compositions, and processes for making such compositions, comprising carrier-free, suspended silver thiosulfate ion complexes in a base were described. Capelli C., U.S. Pat. No. 6,093,414 (hereby incorporated by reference). The compositions of that invention were silver thiosulfate ion complexes that were homogeneously suspended in an anhydrous base. Alternatively, the silver thiosulfate ion complexes of that invention could be incorporated into a matrix and used with a medical device. Pharmaceutical compositions could also be produced, by combining the silver thiosulfate ion complexes with medicinal agents, including, but not limited, to antimicrobial agents, steroids, and anesthetics.

Although the benefit provided by the complexes of the present invention is not limited by an understanding of the precise nature of the complexes, the chemical formula of the primary silver thiosulfate ion complexes formed when a large excess of thiosulfate salt is used is believed represented by Ag(S₂O₃)₃⁵⁻. By comparison, the chemical formula of the primary silver thiosulfate ion complexes formed when only a small excess of thiosulfate salt is used is believed represented by Ag₂(S₂O₃)₃⁴⁻. The preferred silver thiosulfate ion complexes are those represented by Ag₂(S₂O₃)₃⁴⁻. These resulting silver thiosulfate ion complexes are in a relatively pure solid form, and are stable, highly water soluble and antimicrobially active.

The silver compositions of the '414 patent are preferably derived from the complexation of silver cations from silver halides (preferably silver chloride) with anions from the sodium thiosulfate salts. The preferred molar ratio of the thiosulfate anions to the silver cations was at least 1:1 and more preferably at least 1.3:1. It is desirable that the silver thiosulfate ion complexes are solid and essentially pure, i.e., they did not contain significant amounts of waste salts or other substances that interfere with their antimicrobial activity; in addition, they do not require carrier particles.

The silver thiosulfate-ion compositions of the '414 patent are also stable against heat and light. However, in the presence of water, or aqueous containing bases or polymers, these silver thiosulfate-ion compositions degrade over time.

This destabilization of silver thiosulfate occurs when the thiosulfate ion component of the silver thiosulfate ion complexes experiences a chemical breakdown. The effect of this chemical process results the breakdown of the silver thiosulfate ion complex and concomitant loss of antimicrobial activity.

While an understanding of the mechanisms involved is not necessary for successful use of the invention, it is believed that the thiosulfate ion which makes up the silver thiosulfate ion complex (i.e., of the '414 patent) is formed by adding a sulfur atom to a sulfite ion in a complex reaction that can be summarized by the following chemical equation: S+SO₃²⁻=S₂O₃2−(i.e., thiosulfate ion). The sulfur atom that is added to the sulfite ion to give S₂O₃²⁻ is somewhat labile; thus, S₂O₃²⁻ may appropriately be represented as S—SO₃²⁻. In aqueous solutions, the thiosulfate ion decomposes over time. At moderately low pH levels the sulfur atom readily splits off, nominally yielding sulfur as follows:

S—SO₃²⁻+H⁺═S+HSO₃¹⁻

Acid mediated decomposition of the thiosulfate ion nominally yields sulfur, however, it is known that very finely divided particles of sulfur in an acidic aqueous solution have the character of polysulfide ions. Levenson, Complementary Processes, In: “The Theory of the Photographic Process”, Chapter 14, Fourth Ed. MacMillan Publishing Co., Inc., New York (1977).

As a result of aqueous thiosulfate ion instability, aqueous solutions of silver thiosulfate ion complexes chemically decompose over time. One possible explanation is that when the thiosulfate component of the silver thiosulfate ion complex chemically breaks down, silver ions are released which react with the released sulfur ions and form a silver sulfide. Silver sulfide is a black material (i.e., Ag₂S). Due to silver sulfide's high dissociation constant (pK=49.1), silver sulfide has minimal antimicrobial activity. That is to say, the silver ion is bound tightly to the sulfur ion and ionizes very slowly from the silver sulfide salt. As a result, little, if any, ionized silver is available to provide antimicrobial activity.

Silver thiosulfate ion complexes compatible with a sulfite-stabilized preservation technique have previously been disclosed. Capelli C., U.S. Pat. No. 6,093,414 (herein incorporated by reference). These silver thiosulfate ion complexes, when added to either an ointment base which contains a small proportion of water or a water-containing cream base in order to form an antimicrobial composition, will decompose over a relatively short period of time. The resulting antimicrobial composition will turn black as the silver thiosulfate ion complexes in the composition decompose to silver sulfide. Consequently, the composition will lose its antimicrobial efficacy in proportion with the observed decomposition of the silver thiosulfate ion complexes.

Sulfites

The use of sulfites in the formation of antimicrobial silver thiosulfate ion complexes have been disclosed in the prior art. Oka, U.S. Pat. Nos. 5,326,567 and 5,429,819; and Nishino, U.S. Pat. No. 5,510,109. The '567 and '819 patents disclose that thiosulfate metal salt complexes were obtained by adding a salt selected from the group consisting of a sulfite salt and a hydrogen sulfate salt to an aqueous solution of a metal salt and then adding the resulting thiosulfate salt to an aqueous solution. The '109 patent created metal thiosulfate complexes by reacting at least one compound selected from the group consisting of sulfite and bisulfite to an aqueous solution of a metal salt, followed by the addition of thiosulfate; or by adding a metal salt to an aqueous solution of thiosulfate. The silver thiosulfate complexes of the '567, '819 and '109 patents share a major limitation in that all require a porous silica gel particulate carrier to resolve difficulties known in the art when creating carrier-free silver thiosulfate ion complexes.

While the use of sulfite is known to provide added stability to silver thiosulfate complexes, the life-time of carrier-required sulfite stabilized aqueous silver thiosulfate complexes is limited. Although it is not necessary to understand an invention, it is believed that this instability of carrier-required sulfite stabilized aqueous silver thiosulfate complexes is due to the instability of the sulfite ion. Specifically, the sulfite ion rapidly degrades in the presence of oxygen. Consequently, the degradation of the sulfite ion decreases the sulfite's ability to stabilize aqueous silver thiosulfate ion complexes.

It is known that SO₃₂— is converted into SO₄₂— when the medium contains dissolved oxygen, or into SO₂— if the medium is significantly acidic or alkaline. Yagi et al., J. Chromatogr 292:273-380 (1984). The oxidation of SO₃₂— also proceeds in the presence of certain catalysts as traces of the salts of copper or iron. Degradation of SO₃₂ into SO₄₂ in aqueous solution has been shown by capillary zone electrophoresis to occur rapidly over the course of a few days. Carvalhho et al., “Sulfur Speciation By Capillary Zone Electrophoresis: Conditions For Sulfite Stabilization And Determination In The Presence Of Sulfate, Thiosulfate And Peroxodisulfate.” Fresenius J Anal Chem 368:208-213 (2000).

With the degradation of SO₃₂— (i.e., sulfite ion) into SO₄²⁻ (i.e., sulfate ion) the stabilizing effect of a sulfite ion on a silver thiosulfate ion complex is lost. One approach to overcome this problem is to provide a large excess amount of sulfite relative to the aqueous silver thiosulfate ion complex. This approach, however, is less than ideal, if the silver thiosulfate ion complex is intended to contact living tissue. Specifically, high concentrations of sulfite ion cause tissue irritation, and is painful. Additionally, the breakdown of aqueous sulfite ion produces hydrogen sulfide and, consequently, the production of a highly disagreeable odor. Using large amounts of sulfite ion in order to provide thiosulfate stabilization can lead to a product having an odor so strong as to make it un-useable and unmarketable.

Surprisingly, the present invention discloses that silver thiosulfate ion complexes are effectively stabilized by the use of sulfite salts wherein said stabilized silver ion complex composition is carrier-free. Even more surprising, the present invention discloses that sulfite preservative agents improve the stabilization of aqueous silver thiosulfate ion complexes by stabilizing sulfite ions in a salt solution.

One embodiment of the present invention contemplates sulfite stabilizing agents that stabilize carrier-free aqueous silver thiosulfate ion complexes. Preferably, these sulfite stabilizing agents minimize the breakdown of the thiosulfate ligand of the carrier-free aqueous silver thiosulfate ion complex. While an understanding of the mechanisms involved is not necessary, it is believed that the thiosulfate equilibrium with sulfite and sulfide is as follows:

6H⁺+4SO₃²⁻+2S²⁻=3S₂O₃²⁻+3H₂O

With added sulfite, the equilibrium is shifted to the right. Sulfite addition, therefore, stabilizes the thiosulfate ligand of silver thiosulfate ion complexes contemplated by the present invention.

Any sulfite stabilizing agent that is capable of stabilizing a silver thiosulfate ion complex is contemplated by this invention. In one embodiment, a sulfite stabilizing agent is selected from the group consisting of sodium sulfite, potassium sulfite, sodium bisulfite, potassium bisulfite, sodium metabisulfite, and potassium metabisulfite.

In one embodiment, the present invention contemplates that a sulfite stabilizing agent is added to an aqueous solution containing a silver thiosulfate ion complex during the production of the stabilized silver thiosulfate ion complex. Preferably, a sulfite stabilizing agent is added during the second step in a previously disclosed process for making silver thiosulfate ion complex powders. Capelli C., U.S. Pat. No. 6,093,414 (herein incorporated by reference). This particular step involves the addition of a solvent (i.e., acetone, alcohol, THF, etc.) to an aqueous solution containing a silver thiosulfate ion complex and a sulfite stabilizing agent in such a manner as to create a biphasic separation: in this way, the stabilized silver thiosulfate ion complexes separates into one phase. One embodiment of the present invention contemplates adding a sulfite stabilizing agent at this step of production. Preferably, the addition of a sulfite stabilizing agent results in a relatively pure stabilized silver thiosulfate ion complex. Consequently, excess thiosulfate salts, sulfite salts, waste salts, solvent and other contaminants remain in the non-stabilized silver thiosulfate ion complex phase of the biphasic solution.

Alternatively, another embodiment of the present invention contemplates adding a sulfite stabilizing agent to an aqueous solution containing silver thiosulfate ion complex after the silver thiosulfate ion complex has been produced. In either case, the amount of sulfite stabilizing agent added to produce a carrier-free aqueous silver thiosulfate ion complex solution is sufficient to provide long-term thiosulfate ion complex. A preferred amount of sulfite stabilizing agent is approximately between 0.5% to 10%, but preferably from approximately between 1% to 5%.

Although the benefit provided by the complexes of the present invention is not limited by an understanding of the precise nature of the complexes, the chemical formula of the primary silver thiosulfate ion complexes formed when a large excess of thiosulfate salt is used is believed represented by Ag(S₂O₃)₃⁵⁻. By comparison, the chemical formula of the primary silver thiosulfate ion complexes formed when only a small excess of thiosulfate salt is used is believed represented by Ag₂(S₂O₃)₃⁴⁻. The preferred silver thiosulfate ion complexes are those represented by Ag₂(S₂O₃)₃⁴⁻. These resulting silver thiosulfate ion complexes are in a relatively pure solid form, and are stable, highly water soluble and antimicrobially active.

The present invention contemplates teachings that are novel over previous disclosures regarding sulfite stabilized silver-based complexes by use of a sulfite preservative agent. As discussed above, despite the known fact that the use of a sulfite ion adds stability to silver thiosulfate complexes, this stability is limited over time in an aqueous environment. Although it is not necessary to understand an invention, it is believed that this instability of aqueous sulfite-stabilized silver-based complexes in an aqueous solution is due to the instability of sulfite ion itself. Specifically, the sulfite ion is suspected to degrade rapidly when in an oxygenated aqueous solution. The degradation of the sulfite decreases sulfite's ability to stabilize the silver thiosulfate ion complexes in solution. Surprisingly, one embodiment of the present invention contemplates a stabilizing sulfite agent that improves the stability of a carrier-free aqueous silver thiosulfate ion complex. Although it is not necessary to understand an invention, it is believed that by preventing sulfite degradation, sulfite is maintained at a concentration sufficient to provide stability to carrier-free aqueous silver thiosulfate ion complexes.

Sulfite Preservatives

Preservatives for sulfite ion are known in the art to include alcohols, glycerol and triethanolamine. Mechanisms offered to explain the preservation of sulfite agents include the formation of a stable sulfite ion-preservative agent compound, or a simple ionic complex formation via hydrogen bonding. Carvalhho et al., “Sulfur Speciation By Capillary Zone Electrophoresis: Conditions For Sulfite Stabilization And Determination In The Presence Of Sulfate, Thiosulfate And Peroxodisulfate.” Fresenius J Anal Chem 368:208-213 (2000).

One skilled in the art would expect that a mechanism of preservation by a sulfite ion involving the formation of stable compounds or complexes, would make them less capable of stabilizing thiosulfate ligands. It is a surprising and unexpected discovery, therefore, that the present invention contemplates that even though sulfite agents were preserved, these “preserved” sulfite agents are still able to stabilize a carrier-free aqueous silver thiosulfate ion complex.

In one embodiment, the present invention contemplates the use of sulfite preservative agents that stabilize carrier-free aqueous silver thiosulfate ion complexes while still compatible for use in medical products. Preferably, sulfite preservatives include, but are not limited to, alcohols such as ethanol, isopropyl, butanol, glycerols and the like. Alternatively, polymers containing a high number of alcohol groups are considered sulfite preservatives, such as, but not limited to, polyvinyl alcohol. Furthermore, sulfite preservatives contemplated by the present invention include amines such as, but not limited to, triethanolamine or methyl, ethyl, propyl or butyl amine or tri-hydroxymethylaminomethane. Preferably, an amine sulfite preservative comprises tri-hydroxymethylaminomethane.

The present invention contemplates the addition of an amount of sulfite preservative agent to a carrier-free aqueous silver thiosulfate ion complex sufficient to provide long-term stability. In one embodiment, a sulfite preservative agent ranges from 0.1% to 10% of the aqueous solution. In another embodiment, a sulfite preservative agent ranges from 0.2% to 5% of the aqueous solution.

The present invention demonstrates a significant advantage over previous attempts to stabilize aqueous silver thiosulfate complexes driven by mass action that require a large excess amount of sulfite added to a silver thiosulfate complex solution. Stabilization using a sulfite preservative, results in a lower amount of added sulfite stabilizing agent thereby avoiding the known problem of tissue irritation caused by high concentrations of sulfite. A second significant advantage of the present invention over prior attempts to stabilize aqueous silver thiosulfate complexes driven by mass action is the absence of malodorous hydrogen sulfide produced during the breakdown of a sulfite stabilizing agent. Clearly, the preservation of a sulfite stabilizing agent leads to stable carrier-free aqueous silver thiosulfate ion complex compositions that minimize malodor resulting in products that are more acceptable to patients and medical personnel.

Medical Device Coatings

One embodiment of the present invention contemplates a wound dressing comprising a sulfite stabilized carrier-free aqueous silver ion thiosulfate complex. Preferably, materials suitable for incorporation of sulfite silver ion thiosulfate complexes, include, but are not limited to, traditional gauzes and compresses, hydrocolloid dressings or xerogel dressings. In one embodiment, a sulfite stabilized silver ion thiosulfate complex is readily incorporated by dissolution in water and impregnation into dressings (i.e., for example, gauze), or a complex may be introduced as a component of said dressing, (i.e., as a component of an adhesive composition) whose methods of production are well known in the art.

The present invention contemplates a method incorporating a sulfite stabilized silver ion thiosulfate complex into or onto an alginate fiber dressing (or similar dressing) by simply adding the composition to a solution comprising an alginate prior to producing a fibrous material. In one embodiment, introduction of said thiosulfate complex comprises a powder obtained by methods including, but not limited to, grinding a lyophilized or spray-dried material. In another embodiment, a wound dressing adhesive comprises said thiosulfate complex (i.e., into a foam pad adhesive).

The present invention contemplates that sulfite stabilized carrier-free aqueous silver ion thiosulfate complex compositions and formulations thereof may be used for antibacterial, antiviral or antifungal (i.e., antimicrobial) use in the area of human or veterinary medicine. In one embodiment, sulfite stabilized silver ion complexes are incorporated into medical devices including, but not limited to, medical implants, wound care devices, body cavity and personal protection devices, and the like. By way of illustration only, a sulfite stabilized silver ion complex is incorporated with an anhydrous polymer matrix for a catheter (e.g., urinary catheters) coating that is sufficient to prevent infection. Similarly, sulfite stabilized silver ion complexes are useful in cosmetics and personal care products to make them resistant to antimicrobial contamination.

Examples of cosmetics contemplated by the present invention include, but are not limited to, lipsticks and glosses, lip pencils, mascaras, eye liners, eye shadows, moisturizers, liquid and powder makeup foundations, powder and cream blushes, perfumes, colognes, various creams and toners etc.; and assorted applicators like combs, brushes, sponges, and cotton swabs and balls, and examples of personal care products include deodorants, razors, shaving creams, shampoos, conditioners, various hair treatments like mousses and sprays, toothpastes, mouthwashes, dental flosses and tapes, sunscreens, moisturizers, tampons, sanitary napkins, panty shields, diapers, baby wipes, facial tissues, toilet tissues, etc.

Many other types of medical products are suitable for incorporation of the silver compositions of the present invention including, but not limited to, foam or other vaginal inserts for use in the continence care, condoms, male external urine catheters, skin adhesives etc. Furthermore, compositions of the present invention may be used in products not necessarily being in direct contact with the body, such as powders for removal of odor in incontinence pads or for incorporation into ostomy pouches.

The present invention contemplates sulfite stabilized silver ion complex coatings suitable for use with prosthetic implants, permanent sutures or other implantable biodegradable and biocompatible medical devices. In one embodiment, the present invention contemplates a method to coat a medical device with a sulfite stabilized silver ion complexes under conditions such that the risk of infection either during or after surgery is reduced. Preferably, said medical device is used in conjunction with an generalized surgical infection prevention method comprising; introducing sulfite stabilized silver thiosulfate ion complexes in combination with systemic antibiotic prophylactic treatments and antiseptic skin treatments to a surgical field.

Additionally, similar combination treatments are contemplated for implanted medical devices or those used during surgery for a prolonged period of time. The present invention offers an advantageous alternative to known compositions comprising silver as the composition of the present invention has broad antiseptic properties and is highly stable during storage and use.

Still further, the present invention also contemplates a stabilized silver thiosulfate ion composition comprising a silver thiosulfate ion complex that is complexed with a primary, secondary or tertiary amine. In one embodiment, this silver thiosulfate ion amine complex is associated to one or more hydrophilic polymers, said composition having antibacterial, antiviral and/or antifungal activity and is impregnated into a wound dressing, an ostomy appliance, an incontinence device, other medical devices or hydrophilic coatings.

The invention is explained more in detail in the working examples below disclosing embodiments and properties of compositions of the invention. It is evident that many variations may be made without diverging from the invention the scope of which is set forth in the appended claims.

Experimental

In the section below, the following abbreviations apply: L (liters); ml (milliliters); μl (microliters); g (grams); mg (milligrams); μg (micrograms); mol (moles); mmol (millimoles); μmol (micromoles); cm (centimeters); mm (millimeters); nm (nanometers); ° C. (degrees Centigrade); MW and M.W. (molecular weight); N (normal); w/w (weight-to-weight); w/v (weight-to-volume); min. (minutes); Aldrich (Milwaukee, Wis.); Columbus (Columbus Chemical Industries; Columbus, Wis.); No. (number); CFU (colony forming units); PEG (polyethylene glycol); MHM (Mueller Hinton Medium); ZOI (zone of inhibition); ATCC. It is understood that the following are mere illustrative descriptions of specific embodiments of the present invention, are not intended as limiting in any manner.

### EXAMPLE 1

### Process for Making Silver Thiosulfate Ion Complexes

This example illustrates a process for producing silver thiosulfate ion complexes useful for this invention.

The silver thiosulfate ion complexes were produced by first making a silver chloride precipitate in an aqueous (i.e., deionized water) solution (hereafter, “silver chloride precipitate/aqueous solution”). The silver chloride precipitate/aqueous solution was made by mixing 20 ml of 1 mmol/ml silver nitrate (Aldrich) with 22 ml of 1 mmol/ml sodium chloride (Aldrich) in a 500 ml separatory funnel. To the resulting silver chloride precipitate/aqueous solution was added 60 ml of 1 mmol/ml sodium thiosulfate (Columbus). The resulting mixture was agitated by shaking the separatory funnel until all of the silver chloride precipitate was dissolved.

The silver thiosulfate ion complexes produced were separated by adding 200 ml of ethyl alcohol to the funnel. Upon addition of the ethyl alcohol, the solution became cloudy and separated into two separate phases. The two phases were separated using the separatory funnel. The weight of the material in the phase containing the silver thiosulfate ion complexes was approximately 17 g. This phase was then treated by adding 70 ml ethyl alcohol and 40 ml of acetone to make the silver thiosulfate ion complexes essentially anhydrous. After sitting overnight, the silver thiosulfate ion complexes were in the form of a pure, white solid material in the bottom of the container. Thereafter, the solvent was decanted and the white solid was dried in an oven (i.e., for example, at 62° C.) until the solid was able to be ground into a fine white powder using a mortar and pestle. The weight of the dried silver thiosulfate ion complexes was 10.03 g.

### EXAMPLE 2

### Process for Making Stabilized Silver Thiosulfate Ion Complexes

This example illustrates the process for producing a sulfite stabilized silver thiosulfate ion complex useful for this invention, wherein sulfite is added during the production of the silver thiosulfate ion complex.

The silver thiosulfate ion complexes were produced by first making a silver chloride precipitate in an aqueous (i.e., deionized water) solution (hereafter, “silver chloride precipitate/aqueous solution”). The silver chloride precipitate/aqueous solution was made by mixing 20 ml of 1 mmol/ml silver nitrate (Aldrich) with 22 ml of 1 mmol/ml sodium chloride (Aldrich) in a 500 ml separatory funnel. To the resulting silver chloride precipitate/aqueous solution was added 60 ml of 1 mmol/ml sodium thiosulfate (Columbus). The resulting mixture was agitated by shaking the separatory funnel until all of the silver chloride precipitate was dissolved. To the resulting solution was added 60 ml of a 1 mmol/ml sodium sulfite (Aldrich).

The stabilized silver thiosulfate ion complexes produced were separated by adding 200 ml of ethyl alcohol to the funnel. Upon addition of the ethyl alcohol, the solution became cloudy and separated into two separate phases. The two phases were separated using the separatory funnel. This phase was then treated by adding 70 ml ethyl alcohol and 40 ml of acetone to make the silver thiosulfate ion complexes essentially anhydrous. After sitting overnight, the silver thiosulfate ion complexes were in the form of a pure, white solid material in the bottom of the container. Thereafter, the solvent was decanted and the white solid was dried in an oven (i.e., for example, at 50° C.) until the solid was able to be ground into a fine white powder using a mortar and pestle.

### EXAMPLE 3

### Process for Making Stabilized Silver Thiosulfate Ion Complexes

This example illustrates the process for producing a sulfite stabilized silver thiosulfate ion complex useful for this invention, wherein sulfite is added to a solution containing a dissolved silver thiosulfate ion composition.

A silver thiosulfate ion-complex solution was made by dissolving 0.294 mmol (0.160 g) of silver thiosulfate ion complex (nominal M.W. of 537) made according to Example 1 into 10 ml of distilled water. The resulting solution was clear and colorless. To this silver thiosulfate ion-complex solution was added 0.2 g of sodium sulfite (Aldrich). The final solution contained 0.294 mmol of silver thiosulfate ion-complex, 2% sodium sulfite and 5% isopropyl alcohol.

### EXAMPLE 4

### Process for Making Stabilized Silver Thiosulfate Ion Complexes

This example illustrates the process for producing a sulfite stabilized silver thiosulfate ion complex useful for this invention wherein a sulfite stabilizing agent and a sulfite preservative are added to a solution containing a dissolved silver thiosulfate ion composition.

A silver thiosulfate ion-complex solution was made by dissolving 0.294 mmol (0.160 g) of silver thiosulfate ion complex (nominal M.W. of 537) made according to Example 1 into 8.6 ml of distilled water. The resulting solution was clear and colorless. To this silver thiosulfate ion-complex solution was added 0.2 g of sodium sulfite (Aldrich) and 1.4 ml of a 70% isopropyl alcohol solution. The final solution contained 0.294 mmol of silver thiosulfate ion-complex, 2% sodium sulfite and 5% isopropyl alcohol.

### EXAMPLE 5

### A Non-Stabilized Silver Thiosulfate Ion Complex

A silver thiosulfate ion complex was made by dissolving 0.294 mmol (0.160 g) of silver thiosulfate ion complex (nominal M.W. of 537) made according to Example 1 into 10 ml of distilled water. The resulting solution was clear and colorless.

### EXAMPLE 6

### Stability Study

This example compares the stability of the above examples to illustrate the invention.

A sulfite stabilized silver thiosulfate complex made according to either Example 3 or 4 was compared to a non-stabilized silver thiosulfate complex made according to Example 5. The study was performed by adding a 1 ml sample of each tested silver thiosulfate complex into 8 ml vials that were subsequently sealed. During a 50° C. incubation the samples were examined periodically for signs of marked degradation. The term “marked degradation” means that a solution is observed to have a significant amount of black precipitation. Table 1 identifies the nominal times each of the respective samples took before marked degradation was noted. Specifically, the longer the duration before marked degradation appeared, the more stable the silver thiosulfate complex. The results of this study were as follows:

TABLE 1
Stability of Solutions at 50° C.

| | Time to Marked |
|---|---|
| Sample | Degradation |
| Example 5: Non-Stabilized Silver | <24 hours |
| Thiosulfate Ion-Complex Solution | |
| (n = 3) | |
| Example 3: Stabilized Silver Thiosulfate | No Marked Degradation |
| Solution Using Only A Sulfite Stabilizing | after 72 hours |
| Agent | |
| (n = 3) | |
| Example 4: Stabilized Silver Thiosulfate | No Marked Degradation |
| Solution Using A Sulfite Stabilizing | after 72 hours |
| Agent And A Sulfite Preservative | |
| (n = 3) | |

Table 1 demonstrates that the addition of the sulfite stabilizing agent either with or without a sulfite preservative markedly improves carrier-free aqueous silver thiosulfate ion complex stability.

### EXAMPLE 7

### Accelerated Stability Study

This example compares the stability of the above examples to illustrate the invention.

A stabilized silver thiosulfate ion complex made according to Example 3 or Example 4 were compared to a non-stabilized silver thiosulfate complex made according to Example 5. The study was performed by adding 2 ml sample of each tested complex into 8 ml vials that were subsequently sealed. These samples were then placed in a 93° C. oven and observed every five minutes for signs of marked degradation. The term “marked degradation” means that a solution is observed to have a significant amount of black precipitation. Table 2 identifies the nominal times each of the respective samples took before marked degradation was noted. Specifically, the longer the duration before marked degradation appeared, the more stable the silver thiosulfate complex. The results of this study were as follows:

TABLE 2
Stability of Solutions at 93° C.

| | Time to Marked |
|---|---|
| Sample | Degradation |
| Example 5: Non-Stabilized Silver | 10 minutes |
| Thiosulfate Ion-Complex Solution | |
| (n = 2) | |
| Example 3: Stabilized Silver Thiosulfate | No Marked Degradation |
| Solution using Sulfite Stabilizing Agent | Slight precipitation noted |
| (n = 2) | after 35 minutes |
| Example 4: Stabilized Silver Thiosulfate | No Marked Degradation |
| Solution using Sulfite Stabilizing Agent | Slight precipitation noted |
| and Sulfite Preservative | after 45-55 minutes |
| (n = 2) | |

Table 2 demonstrates that by using an accelerated stability protocol the addition of the sulfite stabilizing agent either with or without a sulfite preservative markedly improves carrier-free aqueous silver thiosulfate ion complex stability. Importantly, the data also clearly demonstrates that a sulfite preservative agent provides added stability to the silver thiosulfate ion complex composition.

Results from testing a non-stabilized silver thiosulfate ion complex composition in conjunction with a sulfite preservative alone demonstrate that a sulfite preservative does not provide any added stability to a non-stabilized silver thiosulfate ion complex (data not shown).

### EXAMPLE 8

### Malodor Inhibition

This example demonstrates the use of a sulfite preservative in the prevention the production of malodor resulting from sulfite agent breakdown.

While an understanding of the mechanisms involved is not necessary, it is believed that malodor is the result of hydrogen sulfide production. Preserving the sulfite stabilizing agent (i.e., preventing the breakdown of sulfite into hydrogen sulfide) not only provides greater silver ion thiosulfate stabilization (supra) but also provides a composition that minimizes malodor. This specific advantage results in products that are more acceptable to the consumers (i.e., for example, patients and medical personnel).

A first mixture was generated comprising 4 ml of a 3% sodium bisulfite (Aldrich) distilled water solution was added 120 mg of tri-hydroxymethylaminomethane (i.e., one sulfite preservative; Aldrich). A second mixture was then generated comprising 4 ml of the 3% sodium bisulfite solution without any sulfite preservative. The two samples were then placed in a 5° C. incubator and checked periodically for malodor for a maximum of 96 hours.

The results of this study demonstrated that the 3% sodium bisulfite solution without any sulfite preservative had a slight hydrogen sulfide odor at the very beginning of the study that became progressively stronger during the first 24 hour observation period. The 3% sodium bisulfite solution with the sulfite preservative, however, had no odor at the beginning of the study nor after 96 hours of incubation.

### Example 9

### Antimicrobial Activity

A stabilized silver thiosulfate ion complex made according to Example 3, except that a 1% sodium sulfite solution was used. Additionally, a non-stabilized silver thiosulfate complex made according to Example 5. The in vitro antimicrobial activity of stabilized silver thiosulfate ion complex solution was compared to non-stabilized silver thiosulfate ion complex solution (both having the equivalent of 1% silver nitrate).

Filter paper discs (7 mm diameter) were soaked with either a stabilized silver thiosulfate solution or a non-stabilized silver thiosulfate solution. The antimicrobial studies were performed by first plating S. aureus (ATCC 29213) or E. coli (ATCC 225922) on tryptic soy agar. A disc containing the stabilized thiosulfate solution or the control solution was placed one each of these microbial lawns. As another control, filter discs soaked in 1% silver nitrate solution were also placed on the culture media. The culture plates were incubated at 37° C. overnight. The zone of microbial growth inhibition (ZOI) was measured (in millimeters) from the edge of each filter disc and the results from two trials were averaged. The larger the measured ZOI, the greater the antimicrobial effect. The results of the study are shown below in Table 3.

TABLE 3
Microbial Inhibition Of Stabilized And Non-
Stabilized Silver Thiosulfate Complexes

| | S. aureus | E. coli |
|---|---|---|
| Sample | Zone Of Inhibition | Zone Of Inhibition |
| Silver Nitrate Solution | 1.5 millimeters | 1.0 millimeters |
| Non-Stabilized Silver | 10.5 millimeters | 5.75 millimeters |
| Thiosulfate Complex | | |
| Solution | | |
| Stabilized Silver Thiosulfate | 7.5 millimeters | 15 millimeters |
| Complex Solution | | |

The results from Table 3 illustrate that a stabilized silver thiosulfate ion complex composition of this invention has antimicrobial activity comparable to non-stabilized silver thiosulfate complex composition. The use of stabilizing agents does not affect the antimicrobial activity of a silver thiosulfate ion complex. In comparison, the antimicrobial activity of the silver nitrate solution is significantly less than either the stabilized or non-stabilized silver thiosulfate ion complex compositions.

I claim:

1. A composition, comprising:
a) a silver ion complex;
b) a sulfite capable of stabilizing said complex; and
c) an agent capable of preserving said sulfite.

2. The composition of claim 1, wherein said silver ion complex comprises thiosulfate.

3. The composition of claim 1, wherein said complex is carrier-free.

4. The composition of claim 1, wherein said composition is aqueous.

5. The composition of claim 1, wherein said sulfite is selected from the group consisting of sodium sulfite, potassium sulfite, sodium bisulfite, potassium bisulfite, sodium metabisulfite, and potassium metabisulfite.

6. The composition of claim 1, wherein said sulfite preservative agent is selected from the group consisting of glycerol, methanol, ethanol, propanol, butanol and polyvinylalcohol.

7. The composition of claim 1, wherein said agent is selected from the group consisting of methylamine, ethylamine, propylamine, butylamine and tri-hydroxymethylaminomethane.

8. A composition, comprising:
a) a carrier-free silver thiosulfate ion complex; and,
b) a sulfite capable of stabilizing said complex.

9. The composition of claim 8, wherein said sulfite is selected from the group consisting of sodium sulfite, potassium sulfite, sodium bisulfite, potassium bisulfite, sodium metabisulfite, and potassium metabisulfite.

10. The composition of claim 8, further comprising an agent is selected from the group consisting of glycerol, methanol, ethanol, propanol, butanol and polyvinylalcohol.

11. The composition of claim 8, further comprising an agent is selected from the group consisting of methylamine, ethylamine, propylamine, butylamine and tri-hydroxymethylaminomethane.
